(19) United States
(12) Patent Application Publication
(10) Pub. No.: US 2011/0224743 A1
(43) Pub. Date: Sep. 15, 2011

(54) Smart Multiple Hand-Piece Micro-Current Facial Toner

(76) Inventor: Shannon Gilbreth Britt, Centennial, CO (US)

(21) Appl. No.: 12/629,172

(22) Filed: Dec. 2, 2009

(51) Int. Cl. A61N 1/36

(52) U.S. Cl. 607/2

(57) ABSTRACT

A body-less facial therapy device teaches smart hand-pieces which are directly connected one to the other. The hand-pieces have thereon multiple electrodes which do not move relative to one another.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### CROSS-REFERENCE TO RELATED APPLICATIONS

N/A

### FIELD OF THE INVENTION

This invention relates generally to non-invasive medical devices, and specifically to micro-current facial massage/facial toner devices.

### STATEMENT REGARDING FEDERALLY FUNDED RESEARCH

This invention was not made under contract with an agency of the US Government, nor by any agency of the US Government.

### BACKGROUND OF THE INVENTION

Electrical micro-current stimulation has proven to be an effective method of improving facial skin tone, effective enough that a flourishing industry provides medical treatments and devices which apply micro-current to a human face to improve facial skin appearance.

In general, such devices work by causing a contraction of the muscles of the human face. It will be understood that there are around a dozen groups of facial muscles, with a total number of facial muscles, depending on the method of counting, that may range as high as roughly one hundred. These muscles are crucial to human identity and self-expression, as the face is the primary non-verbal communication device used by human beings. The socially negative effects of aging on the face are the subject of numerous types of treatment, some quite draconian.

The face-lift surgical procedure and the use of BOTOX® to kill muscles and thus generate a different facial expression are both famous and both are regarded as indications of the extremes to which people wilt go to improve their facial appearance.

However, non-invasive electrical methods of providing an improvement in facial appearance are now known and presently being developed. An example of this is the U.S. Pat. No. 4,957,480 in the name of Morenings, entitled METHOD OF FACIAL TONING.

The Morenings patent teaches (see FIGS. 17-19) a device having a body 100 and two electrodes 102, 104, which are separate and thus can move in relationship to one another. The electrodes have no controls thereon, the controls are located upon the body 100. The electrodes are furthermore not connected directly one to another, rather, the electrodes 102, 104 plug into the body 100. The movability of the electrodes in relationship to one another is key to the method of the Mornings patent, as shown by the fact that FIGS. 1 through 16 all deal with different aspects of the relative motion of the two electrodes.

It would be preferable to provide a smaller portable device having no body.

### SUMMARY OF THE INVENTION

### General Summary

The present invention teaches a body-less facial therapy device. Unlike prior art devices which may have variations in the nature of their hand-pieces (such as electrodes worn on the fingers or the like) but which have a bunglesome body unit, the present device eliminates the need for a body at all.

The present device instead has smart hand-pieces which are directly connected one to the other. The smart hand-pieces have controls which a user may access in order to control the functioning of the device. These controls may be anything from buttons and knobs and LEDs to electronic screens and mini-keyboards.

The smart hand-pieces furthermore have electronic controls inside of them. These electronic devices, such as processor units, digital or analog electronics and so on allow the smart hand-pieces to provide micro-current therapy and to control the nature of the therapy, that is, to control such variables as time, amplitude, voltage, energy, frequency, and so on.

The invention may also have more than two hand-pieces.

### Summary in Reference to Claims

It is therefore another aspect, advantage, objective and embodiment of the invention, in addition to those discussed previously, to provide a non-invasive facial toning system comprising:
first and second hand-pieces, the first hand-piece having therein electronic processors and having thereon electronic controls, each hand-piece having projecting therefrom two electrodes, the four electrodes controlled by the electronic control equipment;
the two hand-pieces having a direct connection therebetween, the electronic connection running directly from the first hand-piece to the second hand-piece.

It is therefore yet another aspect, advantage, objective and embodiment of the invention to provide a facial toning system further comprising:
a third hand-piece having a direct connection to another of the first or second hand-pieces, the third hand-piece having therein electronic processors and having therein electronic controls and having projecting therefrom two electrodes controlled by the electronic control equipment.

It is therefore yet another aspect, advantage, objective and embodiment of the invention to provide a facial toning system wherein the direction connection further comprises an electronic cable.

It is therefore yet another aspect, advantage, objective and embodiment of the invention to provide a facial toning system further comprising:
additional electronic processor equipment within the second hand-piece and further electronic controls on the second hand-piece.

It is therefore yet another aspect, advantage, objective and embodiment of the invention to provide a facial toning system further comprising:

information output devices on at least one of the hand-pieces.

It is therefore yet another aspect, advantage, objective and embodiment of the invention to provide a facial toning system wherein the information output devices further comprise audio speaker devices.

It is therefore yet another aspect, advantage, objective and embodiment of the invention to provide a facial toning system wherein the information output devices further comprise LCD screen devices.

It is therefore yet another aspect, advantage, objective and embodiment of the invention to provide a facial toning system wherein the electronic controls further comprise:

a mini-keypad.

It is therefore yet another aspect, advantage, objective and embodiment of the invention to provide a facial toning system consisting of:
first and second hand-pieces, the first having therein electronic processors and having thereon electronic controls, each hand-piece having at least one therapeutic head controlled by the electronic control equipment;
the two hand-pieces having a direct connection therebetween, the electronic connection running directly from the first hand-piece to the second hand-piece.

It is therefore yet another aspect, advantage, objective and embodiment of the invention to provide a facial toning system wherein each one of the therapeutic heads further comprise:

a pair of electrodes fixed in relationship to one another.

It is therefore yet another aspect, advantage, objective and embodiment of the invention to provide a facial toning system wherein the direct connection further comprises:

an electronic cable.

### BRIEF DESCRIPTION OF THE DRAWINGS

FIG. 1 is a top plan form view of a pair of smart hand-pieces having a direct connection therebetween in a first embodiment of the device.

FIG. 2 is a top plan form view of a single one of the smart hand-pieces of the first embodiment opened up to show the interior of two case halves and the electronic devices within.

FIG. 3 is a top plan form view of a second embodiment of the device having four smart hand-pieces having direct connections therebetween.

FIG. 4 is a view of a PRIOR ART micro-current facial device showing clearly the body part, the lack of a direct connection between the simple hand-pieces and the fact that the electronics and controls of the device are contained within the body portion.

FIG. 5 is a bottom view of a third embodiment of the invention showing a case bottom having a recessed charging apparatus.

FIG. 6 is a bottom view of a fourth embodiment of the invention showing a case bottom having a inductance type charging apparatus.

FIG. 7 is a side view of a fifth embodiment of the invention showing a case side having a battery therein.

### INDEX TO REFERENCE NUMERALS

First smart hand-piece 100
Second smart hand-piece 102
Electronic cable direct connection 104
Electrode pair 106
Electronic display 108
Electronic controls 110
Electronic status indicators 112
Electronic audio output 114
Casing front half 120a
Casing rear half 120b
Electronic processor 122
Electronic devices 124
First smart hand-piece 300
Second smart hand-piece 302
Third smart hand-piece 304
Fourth smart hand-piece 306
Direct connection electronic cable 308
Mini-keypad 310
Body 400
Hand piece 402
Hand piece 404
Case bottom 500
Charging aperture 502
Recess 504
Charging apparatus 506
Case bottom 600
Electro/magnetic induction apparatus 602
Case side 700
Battery 702

### DETAILED DESCRIPTION

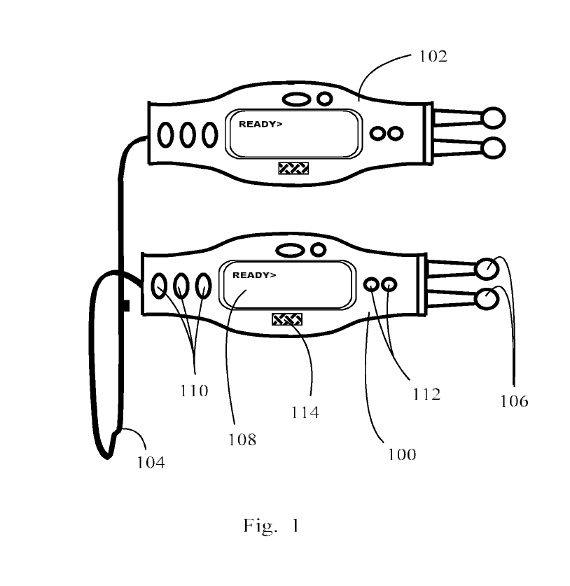

FIG. 1 is a top plan form view of a pair of smart hand-pieces having a direct connection therebetween in a first embodiment of the device.

In the presently preferred embodiment and best mode presently contemplated for carrying out the invention, first smart hand-piece 100 and second smart hand-piece 102 have an electronic cable direct connection 104, which may be differentiated from previous designs by the fact that it does not pass through a control box or body at any point but runs directly from one hand-piece to the other. It will be understood that the smart hand-pieces of the invention permit the elimination of the control box while simultaneously allowing a less expensive, smaller and easier to use device.

Electrode pair 106 on the first hand-piece are fixed in relationship to one another, not moving as in prior art double hand-piece designs. Electronic display 108 may advantageously be a small LCD display screen, such as is shown.

Electronic controls 110 allow the user to input control data in the device and thus control operation. Buttons and so on may be used.

Electronic status indicators 112 may be fairly conventional devices such as LCD lights or the like. Electronic audio output 114 may be a simple buzzer or speaker. These output indicators may be programmed or designed to provide to the user a signal when a complete electrical circuit is made, that is, when current is flowing between electrodes via the skin of the person being treated.

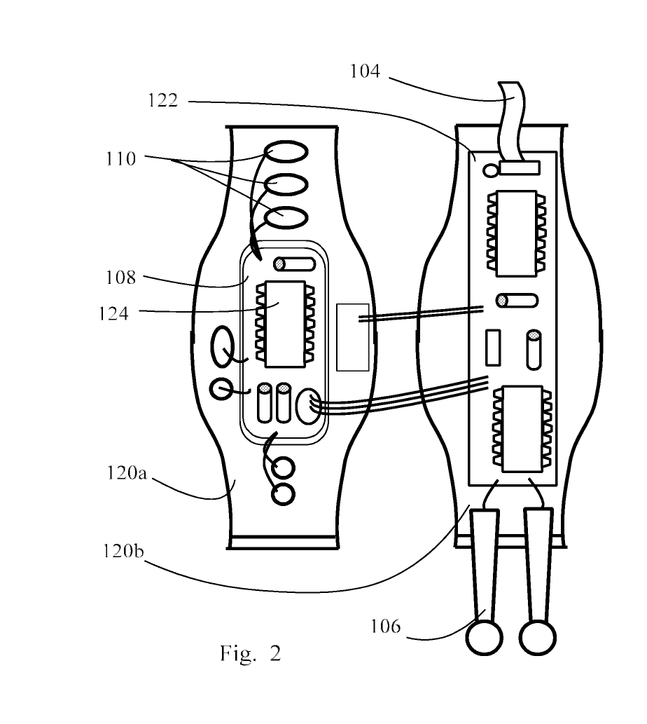

FIG. 2 is a top plan form view of a single one of the smart hand-pieces of the first embodiment opened up to show the interior of two case halves and the electronic devices within.

Casing front half 120a and casing rear half 120b are depicted to open in a clam-shell form but the hand-piece casing may not open or may open in other ways.

Electronic processor 122 and electronic devices 124 cooperate to control the operation of the device, provide the correct wave-forms, micro-current and so on to the electronic heads. As noted, these devices may be digital electronics, analog electronics, IC chips, CPUs and so on.

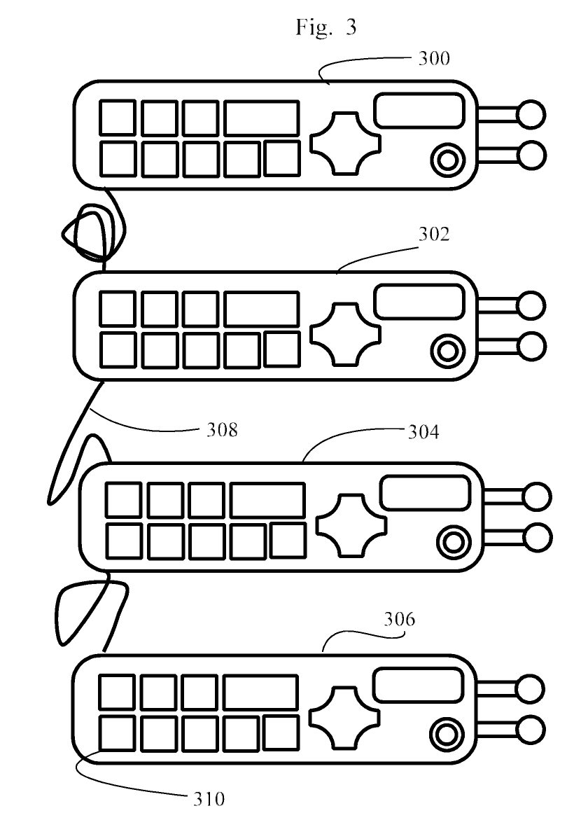

FIG. 3 is a top plan form view of a second embodiment of the device having four smart hand-pieces having direct connections therebetween. First smart hand-piece 300, second smart hand-piece 302, third smart hand-piece 304 and fourth smart hand-piece 306 have a direct connection electronic cable 308. In this embodiment, each hand-piece connects only to one or two other hand-pieces but in other configurations, the cable may connect all four in parallel or the like.

In this embodiment, mini-keypad 310 may be used for more advanced control functions.

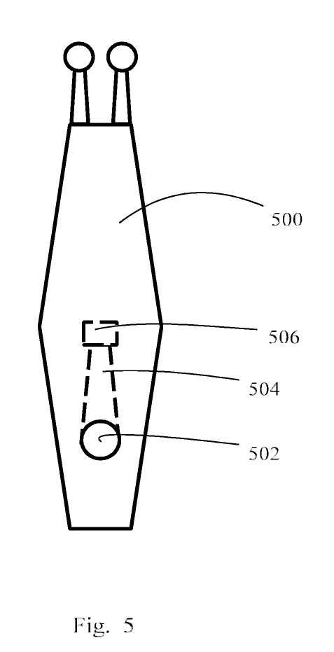

It will be appreciated that control may also be provided by plugging the device into a home computer or docking it with a home computer, as will be discussed below in reference to FIG. 5.

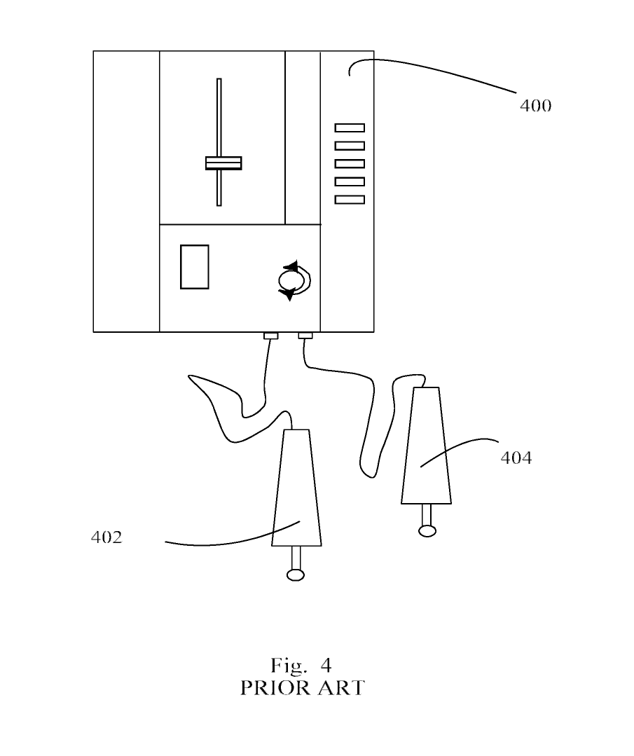

FIG. 4 is a view of a PRIOR ART micro-current facial device showing clearly the body part, the lack of a direct connection between the simple hand-pieces and the fact that the electronics and controls of the device are contained within the body portion. Body 400 is interposed between hand-piece 402 and hand-piece 404, which may be themselves mere electrodes with no electronic functionality whatsoever beyond being simple electrodes with hand grips. Body 400 contains the control apparatus, unlike the present invention.

FIG. 5 is a bottom view of a third embodiment of the invention showing a case bottom having a recessed charging apparatus.

Case bottom 500 has therein charging aperture 502 leading to recess 504, which may have within it charging apparatus 506. A direct electrical connection may be established for charging. It is worth noting that this same apparatus also works quite well for docking to a computer: the charging apparatus 506 may be a simple old fashioned electrical contact or it may be a more modern charging port such as a USB port or the like. It will be appreciated that control may also be provided by plugging the device into a home computer or docking it with a home computer, thus simplifying the type and quantity of input, output, and control electronics necessary in the smart hand-pieces.

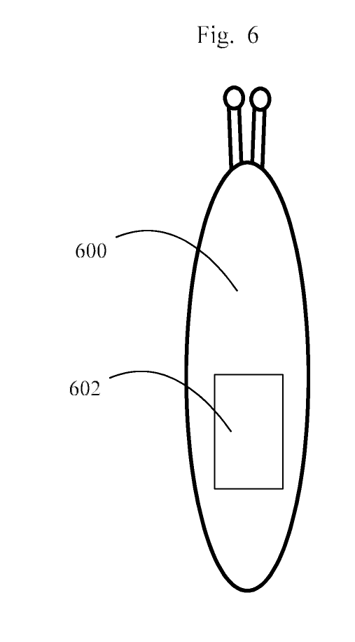

FIG. 6 is a bottom view of a fourth embodiment of the invention showing a case bottom having a inductance type charging apparatus. Case bottom 600 need not be penetrated for electro/magnetic induction apparatus 602 to function: the smart hand-piece may be charged by placing it onto a charging station for inductance of electrical energy.

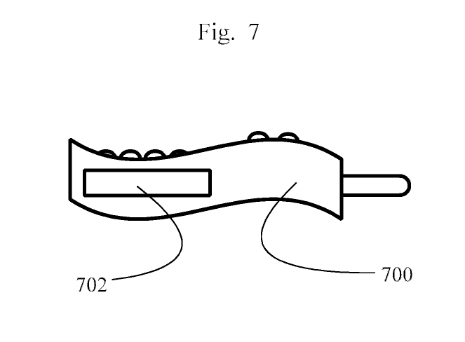

FIG. 7 is a side view of a fifth embodiment of the invention showing a case side having a battery therein.

Case side 700 may have a cover or, as shown, an exposed battery 702.

The disclosure is provided to render practicable the invention by those skilled in the art without undue experimentation, including the best mode presently contemplated and the presently preferred embodiment. Nothing in this disclosure is to be taken to limit the scope of the invention, which is susceptible to numerous alterations, equivalents and substitutions without departing from the scope and spirit of the invention. The scope of the invention is to be understood from the appended claims.

What is claimed is:

1. A non-invasive facial toning system comprising:
first and second hand-pieces, the first hand-piece having therein electronic processors and having thereon electronic controls, each hand-piece having projecting therefrom two electrodes, the four electrodes controlled by the electronic control equipment;
the two hand-pieces having a direct connection therebetween, the electronic connection running directly from the first hand-piece to the second hand-piece.

2. The non-invasive facial toning system of claim 1, further comprising:
a third hand-piece having a direct connection to another of the first or second hand-pieces, the third hand-piece having therein electronic processors and having therein electronic controls and having projecting therefrom two electrodes controlled by the electronic control equipment.

3. The non-invasive facial toning system of claim 1, wherein the direction connection further comprises an electronic cable.

4. The non-invasive facial toning system of claim 1, further comprising:
additional electronic processor equipment within the second hand-piece and further electronic controls on the second hand-piece.

5. The non-invasive facial toning system of claim 1, further comprising:
information output devices on at least one of the hand-pieces.

6. The non-invasive facial toning system of claim 5, wherein the information output devices further comprise audio speaker devices.

7. The non-invasive facial toning system of claim 5, wherein the information output devices further comprise LCD screen devices.

8. The non-invasive facial toning system of claim 5, wherein the electronic controls further comprise:
a mini-keypad.

9. The non-invasive facial toning system of claim 5, wherein the information output devices further comprise indicators which activate to indicate that a complete electrical circuit is made between pairs of electrodes.

10. A non-invasive facial toning system consisting of:
first and second hand-pieces, the first hand-piece having therein electronic processors and having thereon electronic controls, each hand-piece having at least one therapeutic head controlled by the electronic control equipment;
the two hand-pieces having a direct connection therebetween, the electronic connection running directly from the first hand-piece to the second hand-piece.

11. The non-invasive facial toning system of claim 10, wherein each one of the therapeutic heads further comprise:
a pair of electrodes fixed in relationship to one another.

12. The non-invasive facial toning system of claim 15, wherein the direct connection further comprises:
an electronic cable.
